(19) United States
(12) Patent Application Publication
(10) Pub. No.: US 2011/0136768 A1
(43) Pub. Date: Jun. 9, 2011

(54) IMATINIB MESYLATE FOR THE TREATMENT OF NEPHROGENIC SYSTEMIC FIBROSIS

(75) Inventor: Jonathan Kay, Newton Centre, MA (US)

(73) Assignee: THE GENERAL HOSPITAL CORPORATION, Boston, MA (US)

(21) Appl. No.: 12/933,298

(22) PCT Filed: Oct. 22, 2008
(86) PCT No.: PCT/US2008/060678

§ 371 Date: Nov. 17, 2010

Related U.S. Application Data
(60) Provisional application No. 61/037,768, filed on Mar. 19, 2008.

(51) Int. Cl. A61K 31/56; A61K 31/506; A61P 35/00; A61P 17/00; A61P 19/02; A61P 13/12

(52) U.S. Cl. 514/171; 514/252.18

(57) ABSTRACT

Disclosed herein are methods of treatment and/or prevention of nephrogenic systemic fibrosis (NSF) in a subject in need thereof. The methods comprise administering an effective amount of imatinib mesylate to the subject, to thereby treat or prevent the nephrogenic systemic fibrosis. A corticosteroid and/or an antihistamine may also be administered. In one embodiment, the subject has chronic kidney disease, and/or is undergoing hemodialysis, and/or is in renal failure, and/or has been exposed to gadolinium such as gadolinium-containing contrast. The subject may also undergo an additional therapy to treat or prevent nephrogenic systemic fibrosis, chronic kidney disease, or renal failure, such as extracorporeal photopheresis, or administration of pentoxyphyline. Administration may be systemic, such as oral. Useful doses are from about 200 mg-600 mg, a day. Also disclosed are pharmaceutical compositions comprising imatinib mesylate and a pharmaceutically acceptable carrier, optionally formulated for oral administration.

### CROSS-REFERENCE TO RELATED APPLICATIONS

This application claims benefit of U.S. provisional application No. 61/037,768, filed Mar. 19, 2008, the contents of which are incorporated herein by reference in their entirety.

### FIELD OF THE INVENTION

The invention relates to methods of treatment and therapeutic compositions for treatment of nephrogenic systemic fibrosis (NSF).

### BACKGROUND OF THE INVENTION

Nephrogenic systemic fibrosis (NSF) is a devastating, relentlessly progressive condition that lacks effective therapy. Previously called nephrogenic fibrosing dermopathy (NFD) (S E Cowper, H S Robin, S M Steinberg, L D Su, S Gupta, P E LeBoit. Lancet. 2000; 356:1000-1), NSF is an extremely disabling and often painful condition that affects up to 13% of individuals with chronic kidney disease (CKD) (D J Todd, A Kagan, L B Chibnik, J. Kay. Arthritis Rheum. 2007; 56:3433-41). Patients with NSF develop rapidly progressive skin hardening, tethering, and hyperpigmentation, predominantly on the extremities. Flexion contractures of the fingers, elbows, and knees develop in advanced NSF and may severely impair physical function, including ambulation. Cases of NSF with visceral involvement also have been described.⁴⁻⁸ Extracutaneous fibrosis, involving the heart, lungs, diaphragm, skeletal muscle, liver, genitourinary tract, and central nervous system, also occurs in NSF. Renal failure is a common feature in all individuals with NSF, but reports of small case series have not suggested that the risk of developing NSF is associated with a patient's age, gender, race, or cause of renal disease.^{10, 11}

The development of NSF is strongly associated with prior exposure to gadolinium-containing contrast media (OR 14.7 (95% CI [1.9-117.0]) (D J Todd, A Kagan, L B Chibnik, J. Kay. Arthritis Rheum. 2007; 56:3433-41). Gadolinium has been detected in skin biopsies from patients with NSF (High et al., J Am Acad Dermatol. 2007; 56:21-6). The U.S. Food and Drug Administration (FDA) has asked manufacturers to include a new boxed warning on the product labeling of all gadolinium-based contrast agents which are used to enhance the quality of magnetic resonance imaging (MRI). Gadolinium is also used in other imaging (X-ray, as a scintillator in medical imaging equipment, and for detecting neutrons. Gadolinium is also used in nuclear marine propulsion systems, in some nuclear reactors, in various metal alloys, and as a substrate material for magneto-optical films. Gadolinium compounds are used for making phosphors for color television tubes, in the manufacture of compact discs and computer memory, and in masers. It can also be used as a magnetic component for sending hot and cold. Exposure to gadolinium in any of these contexts is thought to increase a subject's risk of developing NSF.

The clinical presentation of NSF is similar to that of other fibrosing disorders and most closely resembles scleromyxedema. However, the clinical features of NSF differ from those of scleromyxedema in that the extracutaneous visceral mucin deposition and associated IgG lambda paraproteinemia of scleromyxedema are absent in NSF.¹ The unique histological appearance of NSF also distinguishes it as a discrete entity. Biopsies of early NSF skin lesions demonstrate thickened collagen bundles, mucin deposition, angiogenesis, and numerous dermal spindle cells that stain with antibodies to CD34 and procollagen.^{11, 15, 16}

NSF appears to be a prevalent complication of renal failure with increased early mortality that represents an emerging epidemic among individuals receiving hemodialysis, especially those who have had exposure to gadolinium. Cutaneous changes of NSF are present in up to 13% of individuals receiving hemodialysis. Among those patients with clinical evidence of NSF, 24-month mortality is increased significantly as compared to those without (48% versus 21%, respectively), with an adjusted hazard ratio of 2.9 (95% CI (1.4-5.9). ¹⁴

Most treatment modalities attempted, such as topical steroids, oral steroids, immunosuppressive therapy, and plasmapheresis, have failed to improve the skin changes of NSF.² Decreased skin thickening and improved joint mobility has been reported in three patients treated with extracorporeal photopheresis¹⁷ and in two other patients treated with pentoxyphyline.¹²

### SUMMARY OF THE INVENTION

Aspects of the present invention relate to a method of treatment and/or prevention of nephrogenic systemic fibrosis (NSF) in a subject in need thereof. The method comprises administering an effective amount of imatinib mesylate to the subject, to thereby treat or prevent the nephrogenic systemic fibrosis. The method may further comprise selecting a subject that has been diagnosed with NSF, has one or more symptoms associated with NSF, or is at increased risk for NSF, prior to administering the imatinib mesylate. Symptoms associated with NSF useful for diagnosis are rapidly progressive skin hardening, skin tethering, hyperpigmentation, joint flexion contracture, extracutaneous fibrosis involving the heart, lungs, diaphragm, skeletal muscle, liver, genitourinary tract, or central nervous system, and combinations thereof. In one embodiment, the subject has chronic kidney disease, is undergoing hemodialysis, is in renal failure, has been exposed to gadolinium, or any combination thereof. In one embodiment, the gadolinium is in the form of a gadolinium-containing contrast. In one embodiment, the subject also undergoes another therapy to treat or prevent nephrogenic systemic fibrosis, chronic kidney disease, or renal failure, such as extracorporeal photopheresis or administration of pentoxyphyline. In one embodiment, treatment of the NSF by one or more of the methods described herein results in softening of previously hardened skin, and/or increased mobility of skin, and/or improved joint mobility.

In one embodiment, the methods described herein further comprise administration of a corticosteroid and/or an antihistamine. In one embodiment, administration of imatinib mesylate, or other agents described herein, is systemic (e.g., oral). Administration of imatinib mesylate can be between about 200 mg and 600 mg of imatinib mesylate administered daily, or about 600 mg, 400 mg, or 200 mg of imatinib mesylate is administered daily.

Aspects of the present invention also relate to a pharmaceutical composition comprising imatinib mesylate and a pharmaceutically acceptable carrier. The pharmaceutical composition may be formulated for oral administration. It may contain about 100-800 mg imatinib mesylate, or about 200-600 mg imatinib mesylate, or about 200-400 mg imatinib mesylate, for example, about 200 mg, 400 mg, 600 mg, or 800 mg imatinib mesylate.

### BRIEF DESCRIPTION OF THE DRAWINGS

FIG. 1(A, B, C; and D) is a set of four line graphs, each representing data reflecting recovery by patients treated with IM. FIGS. 1A and B: represents the change in modified Rodnan skin score (mRSS), over time, in Patients 1 (FIG. 1A) and 2 (FIG. 1B) with IM treatment. FIG. 1C and FIG. 1D: represents the change in knee joint flexion contractures, over time, in Patients 1 (FIG. 1C) and 2 (FIG. 1D) with IM treatment. IM daily dose is indicated on the solid bar above each line graph.

FIG. 2 (A and B) are photographs of skin biopsies from Patient 1 before (FIG. 2A) and after 15 weeks (FIG. 2B) of treatment with IM, at similar magnification and under equivalent lighting. A comparison of the two photos demonstrated different tinctorial qualities indicative of lesser fibrosis after treatment. (hematoxylin & eosin staining, 12.5× total magnification). The grey line in each panel identifies the level of the eccrine ducts, structures that occur at a relatively uniform depth in a particular region of the body.

FIG. 3 (A and B) are photographs of skin biopsies from Patient 1 before (FIG. 3A) and after 15 weeks (FIG. 3B) of treatment with IM, at similar magnification and under equivalent lighting. A comparison of the two photos demonstrated lesser overall staining for type I procollagen and fewer type I procollagen+fibrocytes, particularly in the upper dermis, after treatment. (type I procollagen immunohistochemical staining, 12.5× magnification) The grey line in each panel identifies the level of the eccrine ducts.

### DESCRIPTION OF THE INVENTION

One embodiment of the present invention relates to a method of treatment of nephrogenic systemic fibrosis (NSF) in a subject in need thereof, comprising, administering an effective amount of imatinib mesylate to the subject, to thereby treat the NSF.

Subjects in need of such treatment with this method are those who have been diagnosed with NSF, or who exhibit one or more symptoms associated with NSF, or subjects who are at risk for NSF but have not been examined for NSF or symptoms thereof. In one embodiment, the method further comprises selection of such a subject as suitable for treatment, as described herein, prior to administering the imatinib mesylate. Such a subject is selected on the basis of known exposure, physical condition (e.g., diagnosed, suspected, or anticipated kidney disfunction), diagnosis (e.g. exhibition of one or more symptoms, biopsy, etc.).

The term “treatment” is used to refer to treating a condition that has already manifested in the subject. Manifestation would be by the appearance of one or more symptoms of NSF, such as rapidly progressive skin hardening, skin tethering, hyperpigmentation, joint flexion contracture, and extracutaneous fibrosis involving the heart, lungs, diaphragm, skeletal muscle, liver, genitourinary tract, or central nervous system. Treatment is also used to refer to a slowing of onset and/or severity of additional symptoms wherein the subject already has one or more symptoms. As such, subjects suitable for treatment include those who exhibit one or more symptoms of NSF and are at risk for developing additional symptoms of NSF. Such subjects also include those with one or more symptoms of NSF, but who have not been diagnosed with NSF.

Successful treatment is evidenced by amelioration of one or more symptoms, including softening of previously hardened skin, increased mobility of skin, and improved joint mobility. Acceptable methods of assessment are known to the skilled practitioner. For example, increased mobility (also referred to as decreased skin tethering) can be assessed by skin tethering measurements before and after treatment, by a modified Rodnam skin score. Increased joint mobility (e.g., knee and elbow joints) can be assessed by measurements before and after treatment using a goniometer. The histologic appearance of skin biopsy may also change as a result of successful treatment. A change in visual analog scale (VAS) for pain of the subject may change, as may the change in a health assessment questionnaire (HAQ) score. In one embodiment, improvements are expected to be detectable within 2 months from initiation of treatment.

Administration of imatinib mesylate to a subject is expected to prevent or retard the development of NSF in the subject. As such, one aspect of the present invention relates to a method of prevention of nephrogenic systemic fibrosis (NSF) in a subject comprising, administering an effective amount of imatinib mesylate to the subject, to thereby prevent the nephrogenic systemic fibrosis.

The term “prevention” is used to refer to a situation wherein a subject does not yet have a condition which has manifested in any appreciable form. Prevention encompasses prevention or slowing of onset and/or severity of a symptom, (including additional symptoms wherein the subject already has one or more symptoms). Prevention is performed generally in a subject who is at risk for development of NSF. Such subjects are said to be in need of prevention, and include those who have been exposed to gadolinium (e.g., a gadoloinium-containing contrast), or who are undergoing hemodialysis, or who suffer from kidney disease (chronic or acute) or are in renal failure, or any combination of these conditions. In one embodiment, the method of prevention, described herein, further comprises selection of such a subject at risk for NSF or development of symptoms thereof, as described herein, prior to administering the imatinib mesylate.

### NSF

NSF, also referred to in the art as nephrogenic fibrosing dermopathy (NFD) is typically characterized by skin hardening, tethering (which can be rapidly progressive), swelling and tightening of the skin, usually limited to the extremities, but sometimes involving the trunk. The condition may develop over a period of days to several weeks. In many cases, skin thickening inhibits the flexion and extension of joints, resulting in painful contractures, especially in the fingers, elbows, and knees, which can severely impair physical function. In the most severe of cases, affected patients may be unable to walk, or fully extend the joints of their arms, hands, legs, and feet. Cases of NSF with visceral involvement also have been described.⁴⁻⁸ Extracutaneous fibrosis, involving the heart, lungs, diaphragm, skeletal muscle, liver, genitourinary tract, and central nervous system, also occurs in NSF. Complaints of muscle weakness are common. Once a patient contracts NSF/NFD, the skin changes, including hyperpigmentation, such as reddened or darkened patches, papules, or plaques. In time, the skin surface may distort to resemble the texture of the peel of an orange. Some patients may experience burning, itching, or severe sharp pains in areas of involvement. Radiography may reveal calcifications of the soft tissue. NSF/NFD patients can report “bone ache” described in the hips and in the ribs. Topically, the skin lesions are commonly symmetrical, with zones between the ankles and thighs most commonly involved, followed by involvement between the wrist and upper arms. Hand and foot swelling with blister-like lesions has also been reported in patient with NSF/NFD. Some patients have reported yellow papules or plaques on or near the eyes. A patient may be treated at any point in the onset of NSF, e.g. at the manifestation of any one or more of NSF symptoms such as these.

### Gadolinium

As discussed above, exposure to gadolinium greatly increases a subject's risk for developing NSF. Gadolinium is a silvery white, malleable and ductile rare earth metal with a metallic luster. Gadolinium is also called gadolinium-DPTA and gadodiamide, and it goes by various brand names. Gadolinium is non-radioactive and in commonly used solutions resembles plain water.

Because of their paramagnetic properties, solutions of organic gadolinium complexes and gadolinium compounds are used as intravenous radiocontrast agents to enhance images in medical magnetic resonance imaging. Gadolinium-based contrast agents, also referred to herein as gadolinium-containing contrasts, are commonly used to improve the visibility of internal structures when patients undergo an MRI. Five have been approved for use in the United States: Magnevist (gadopentetate dimeglumine), Ominiscan (gadodiamide), OptiMARK (gadoversetamide), MultiHance (gadobenate dimeglumine), and Prohance (gadoteridol).

Besides MRI, gadolinium (Gd) is also used in other imaging. In X-ray, gadolinium is contained in the phosphor layer suspending in a polymer matrix at the detector. Gadolinium oxyorthosilicate (Gd2SiO5, GSO; usually doped by 0.1-1% of Ce) is a single crystal that is used as a scintillator in medical imaging equipment like as Positron Emission Tomography (PET), and for detecting neutrons. Due to extremely high neutron cross-section of gadolinium, this element is very effective for use with neutron radiography.

Gadolinium is also used in nuclear marine propulsion systems as a burnable poison. Gadolinium is also used as a secondary, emergency shut-down measure in some nuclear reactors, particularly of the CANDU type. Gadolinium is also used in various metal alloys to improve the workability and resistance of iron, chromium and related alloys to high temperatures and oxidation. Gadolinium gallium garnet (Gd3Ga5O12) is a material with good optical properties, and is used in fabrication of various optical components and as substrate material for magneto-optical films. Gadolinium is also used for making gadolinium yttrium garnets, which have microwave applications, and gadolinium compounds are used for making phosphors for color TV tubes. Gadolinium is also used for manufacturing compact discs and computer memory. Gadolinium ethyl sulfate, which has extremely low noise characteristics, may also be used in masers. Furthermore, gadolinium's high magnetic moment and low Curie temperature (which lies just at room temperature) suggest applications as a magnetic component for sensing hot and cold. Exposure to gadolinium in any of these contexts may increase a subject's likelihood/risk of developing NSF. In one embodiment, the exposure detectably increases the gadolinium in the subject's circulatory system. This risk is enhanced considerably if the subject has any form of kidney dysfunction such as acute or chronic kidney disease.

In one embodiment of the invention, the subject is also undergoing another therapy. Such therapies include, without limitation, other therapies to treat or prevent NSF, therapies to treat or prevent kidney disease (chronic or acute), or therapies for subjects in renal failure. Other such therapies include, without limitation, extracorporeal photopheresis, administration of pentoxyphyline, use of topical steroids, administration of oral steroids, immunosuppressive therapy, and plasmapheresis. In one embodiment, the subject is further administered corticosteroid and/or antihistamine.

As used herein, the term “effective amount” or “therapeutically effective amount” as used in conjunction with administration of imatinib mesylate refers to an amount of the active imatinib mesylate in the administered formulation, at dosages and for periods of time necessary, to achieve the desired result, such as sufficient to produce an amelioration of one or more symptoms of NSF described herein, in a subject. An effective amount of the active compound as defined herein may vary according to factors such as the disease state, age, and weight of the subject, and the ability of the active compound to elicit a desired response in the subject and can be determined by the skilled practitioner. Dosage regimens may be adjusted to provide the optimum therapeutic response. An effective amount is also one in which any toxic or detrimental effects of the active compound are outweighed by the therapeutically beneficial effects.

A therapeutically effective amount or dosage of the imatinib mesylate may range from about 100-800 mg, with ranges of the invention including about 200-400 mg. Administration can be daily, every other day, or every 3, 4, 5, 6 days, or weekly. Preferably, administration is daily. However, upon amelioration of symptoms, it may be useful to administer less frequently unless symptoms re-emerge.

In a further embodiment, the subject is administered between about 200 mg to about 600 mg of imatinib mesylate daily. In one embodiment, the subject is administered either about 600 mg, 400 mg, or 200 mg daily. The skilled artisan will appreciate that certain factors may influence the dosage required to effectively treat a subject, including but not limited to the severity of the symptoms, previous treatments, the general health and/or age of the subject, and other diseases present. Moreover, treatment of a subject with a therapeutically effective amount of the active compound can include a single treatment or a series of treatments. In one example, a subject is treated with an active compound in the range of between about 200-400 mg daily, for between about 1 to 10 weeks, alternatively between 2 to 8 weeks, between about 3 to 7 weeks, or for about 4, 5, or 6 weeks. In some cases, prolonged, indefinite treatment (e.g. for months at a time, 1, 2, 3, 4, etc. 6 months or longer) will be optimal. In some circumstances, the subject should undergo treatment until amelioration of symptoms, with cessation of treatment, and re-initiation of treatment if and when symptoms again manifest. It will also be appreciated that the effective dosage of an active compound used for treatment may increase or decrease over the course of a particular treatment. It may be necessary to adjust dosage when the subject is exposed to drugs that alter imatinib mesylate plasma concentrations, such as inhibitors of cytochrome P450 isoenzyme (CYP3A4) which are expected to increase imatinib mesylate concentrations. Because warfarin is metabolized by CYP2C9 and CYP3A4, patients who require anticoagulation should receive standard heparin or monitor closely PT/INR on warfarin while on imatinib mesylate

The skilled practitioner will recognize that the dose amounts and frequency of administration can be changed over the course of the regimen, especially as symptoms become alleviated or increase. The regimen can be for weeks or months, continual, intermittent, temporary or permanent, with determination on an individual basis by the skilled practitioner.

In one embodiment, the imatinib mesylate is an ingredient (e.g., the active ingredient) in a pharmaceutically acceptable formulation suitable for administration to the subject. Generally this comprises a pharmaceutically acceptable carrier for the active ingredient. The specific carrier will depend upon a number of factors (e.g., the route of administration).

Administration to the subject can be either systemic or localized. This includes, without limitation, dispensing, delivering or applying an active compound (e.g. in a pharmaceutical formulation) to the subject by any suitable route for delivery of the active compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

The term “subject” is intended to include animals. In particular embodiments, the subject is a mammal, a human or nonhuman primate, a dog, a cat, a horse, a cow or a rodent.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Example 1

To assess its effect on skin involvement and joint contractures, two patients with NSF were treated with imatinib mesylate. Two patients diagnosed with NSF that was associated with gadolinium exposure were assessed and treated with orally administered imatinib mesylate over an extended period of time. Their progress was assessed as were any side effects of the treatment.

Patient 1: A 75-year-old Caucasian man, underwent a right nephrectomy at age 53 to treat renal cell carcinoma. His renal function gradually declined and hemodialysis (HD) was initiated at age 74. In July 2005, two months after beginning HD, he underwent MR angiography (MRA) of both legs with gadolinium-containing contrast (gadodiamide). One year later (August 2006), following staphylococcal infection of a dialysis catheter in his right upper chest, he developed posterior neck pain that was evaluated by MRI of his cervical spine with gadolinium-containing contrast (gadodiamide). Staphylococcus aureus discitis of the C3-C4 intervertebral disc was diagnosed and he was treated with external immobilization of his cervical spine and 12 weeks of intravenous vancomycin and oral rifampin. To monitor his progress while on antibiotic therapy, he underwent two subsequent MRIs using gadolinium-containing contrast (gadodiamide).

Within one month, he began to experience stiffening of his hands and feet. By September 2006, the skin on his hands and legs had become harder and darker in color than his skin elsewhere. Over the subsequent three months, these skin changes progressed, early contractures of the fingers, elbows and knees were noted (he became unable to fully extend the fingers of either hand or either elbow or knee). He could not walk and was confined to a wheelchair or bed. Biopsy of skin from his left lower leg, performed in October 2006, and demonstrated fibrosis, mucin deposition, and infiltration of CD34+, type I procollagen+, CD68+, and factor XIIa+cells. Measurement of gadolinium in this same tissue demonstrated 130 ppm (μg/g, dry weight) of gadolinium (WA High et al.: Gadolinium is quantifiable within the tissue of patients with nephrogenic systemic fibrosis, J Am Acad Dermatol 2007).

Because of the severity of his skin involvement, his physical disability, and the lack of a consistently effective treatment for NSF, he was offered a four-month course of imatinib mesylate. After providing informed consent, he began treatment with oral imatinib mesylate 400 mg daily in November 2006. Administration of imatinib mesylate was discontinued at week 15. Administration of imatinib mesylate was re-initiated, at 200 mg/day, at 24 weeks.

Patient 2: A 65-year-old Caucasian man with type 2 diabetes mellitus, hypertension, and atrial fibrillation requiring Coumadin® anticoagulation, developed chronic kidney disease at age 59 and peritoneal dialysis (PD) was initiated at age 62. In February 2005, he underwent MRA of his aorta with gadolinium-containing contrast (gadopentetate dimeglumine). One year later, he began to experience stiffness and to note tightening and thickening of the skin over each lower leg and on his fingers. Biopsy of skin on his right lower leg, in October 2006, demonstrated dermal fibrosis and septal widening, mucin deposition, and infiltration of CD34+, type I procollagen+, CD68+, and factor XIIa+cells, consistent with NSF. Measurement of gadolinium in this tissue demonstrated 28 ppm (μg/g, dry weight) of gadolinium (WA High et al.: Gadolinium is quantifiable within the tissue of patients with nephrogenic systemic fibrosis, J Am Acad Dermatol 2007).

He was offered treatment with imatinib mesylate. After providing informed consent, he began taking oral imatinib mesylate 400 mg daily in December 2006. Shortly thereafter, his PD catheter stopped functioning and he began to undergo HD.

To determine whether a higher dose of IM might yield further improvement in his skin changes, his daily dose was increased from 400 mg to 600 mg at the end of January 2007. Because of a hospitalization in February 2007, for evaluation and treatment of chronic gastrointestinal bleeding, he stopped taking IM for 2 weeks. He resumed taking daily oral IM 600 mg, one week later, but he again stopped taking it after 6 days, when he underwent elective surgical repair of an abdominal hernia. He once again resumed taking oral IM 400 mg daily in May 2007. However, because of difficulty controlling his INR, he discontinued IM two weeks later. Off of IM, his Coumadin anticoagulation has been managed more easily.

### Results

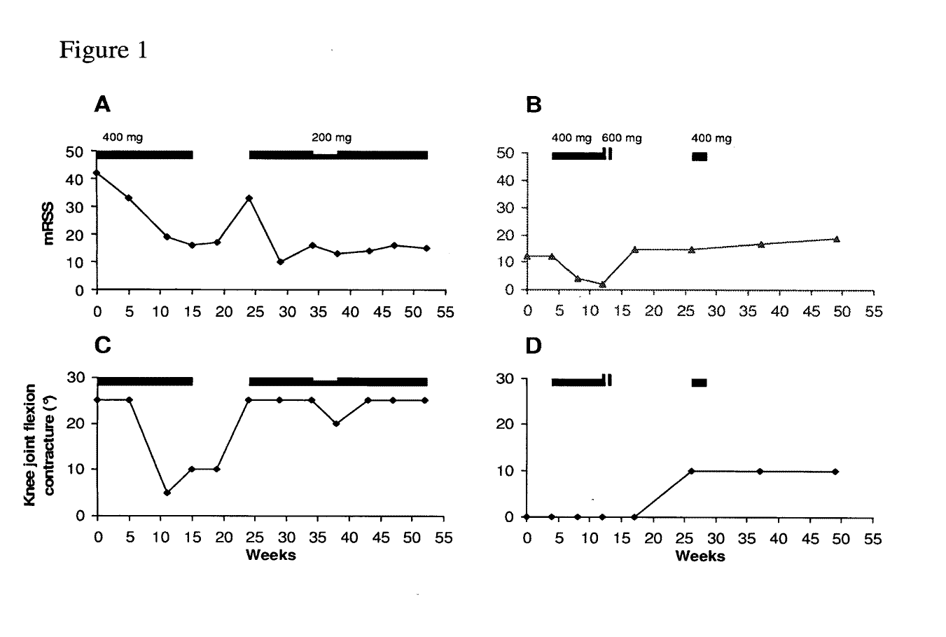

Following the initiation of imatinib mesylate administration, each patient displayed progressive reduction of skin thickening and tethering with a steady decrease in the mRSS (FIG. 1, A and B). While receiving oral IM 400 mg daily over 15 weeks, Patient 1 experienced a decrease in his mRSS from 42 to 16. (FIG. 1A) Over the subsequent 9 weeks, during which he took no IM, his mRSS increased to 33. While taking oral IM 400 mg daily, Patient 2 also exhibited a reduction in his mRSS from 12 to 2. (FIG. 1B) Within 5 weeks of stopping IM, his skin changes recurred and extended to involve his abdomen, resulting in an mRSS of 15.

Because of worsening skin changes that developed off treatment, Patient 1 requested that IM be restarted. Within 5 weeks of resuming oral IM 400 mg daily, his mRSS decreased from 33 to 10 and remained in that range with continued treatment. (FIG. 1A) Patient 2 elected to remain off of IM, other than for a brief two week period, and he experienced persistence of and slight progression of his skin thickening and tethering over the subsequent 20 weeks. (FIG. 1B)

As his mRSS decreased, and with physical therapy employing passive range-of-motion exercises, Patient 1 experienced reduction of his knee joint flexion contractures from 25° at week 6 to 10° at week 15 and he was able to walk about 20 feet with the assistance of a walker. (FIG. 1C) This improvement persisted during the first 5 weeks after he stopped taking IM. However, after he had been off of IM for 9 weeks, he again developed 25° flexion contractures of both knees. Because of medical issues unrelated to his NSF, he did not receive regular physical therapy after reinitiating IM, yet he remains able to walk with the assistance of a walker. Patient 2 had no knee joint flexion contractures when he began taking IM. (FIG. 1D) However, when his skin changes began to progress after discontinuing IM, knee joint flexion contractures developed that gradually progressed over time.

Other than fluid retention, successfully corrected by dialysis treatment, neither patient experienced any adverse effect related to IM. Patient 1 developed gastrointestinal upset that resolved when corticosteroid replacement with 7.5 mg/day oral prednisone was initiated to treat adrenal insufficiency 17 weeks after re-initiation of IM, despite continuation of IM. Neither patient developed congestive heart failure.

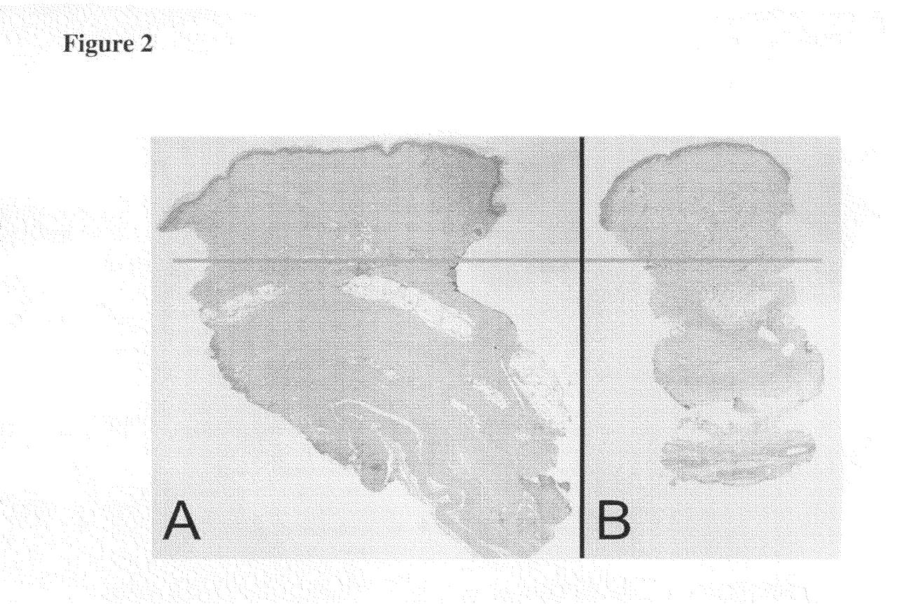

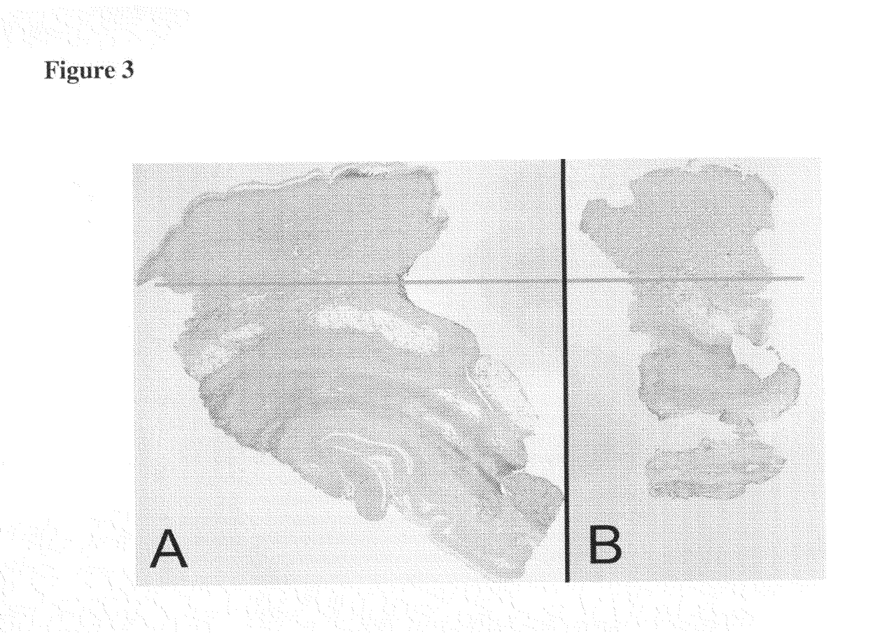

Skin biopsies performed both before and after initial dosing of Patient 1 with IM were compared. Both specimens were obtained from the same anatomic area, with the second biopsy taken immediately adjacent to the resolved scar from the prior procedure, but each biopsy had been performed using different technique (The first biopsy was an excisional biopsy, the second was a punch biopsy). Despite this limitation, when tissue sections of the same thickness (3.5 μm), stained at the same institution in sequential fashion on the same day, were compared side-by-side using the same microscope and under identical lighting, the specimen taken after 15 weeks of IM dosing revealed less fibrosis (FIGS. 2A & 2B) and less staining for type I procollagen (FIGS. 3A & 3B), which correlates with the formation of new collagen (Riaz et al., J Clin Pathol 1994, 47:892-9). Furthermore, in the specimen obtained after IM treatment, fewer type I procollagen⁺ cells were present in the upper dermis (FIG. 3B). The second biopsy contained 124 ppm (μg/g, dry weight) of gadolinium, essentially unchanged from that of the pretreatment biopsy (High et al.: Gadolinium is quantifiable within the tissue of patients with nephrogenic systemic fibrosis, J Am Acad Dermatol 2007).

### Discussion

IM treatment resulted in the relatively rapid and steady improvement of skin thickening and tethering in two patients with stage 5 CKD and NSF. The patient who had knee joint flexion contractures achieved reduction of his flexion contractures with passive range-of motion exercises, once his skin thickening and tethering had begun to decrease. Skin gadolinium content did not decrease significantly with IM treatment but, upon histologic examination of skin, decreased fibrosis and type I procollagen staining correlated with the observed clinical improvement. In each patient, after discontinuation of IM, skin changes progressed within 1 to 2 months and knee joint flexion contractures also worsened or developed de novo. Strikingly, recurrent skin thickening and tethering improved in the patient who resumed taking IM for longer than two weeks. The recurrence of skin thickening and tethering after discontinuation of IM and the response to re-treatment suggest a direct relationship between the administration of IM and improvement in NSF skin changes.

IM is metabolized primarily in the liver and excreted in bile. Dosing need not be adjusted in stage 5 CKD (Pappas et al., Cancer Chemother Pharmacol 2005, 56:358-60). When used to treat CML and GIST, side effects of IM are typically mild-to-moderate in severity and generally resolve after the initial two months of treatment. The most common side effects include fluid retention, nausea, and muscle cramps (Savage et al., N Engl J Med 2002, 346:683-93). Several patients treated with IM for hematologic malignancy have developed new onset congestive heart failure (CHF), but the incidence of CHF among patients treated with IM in clinical trials was not increased above that in the general population (Kerkela et al., Nat Med 2006, 12:908-16; Atallah et al., Blood 2007, 110:1233-7).

Among its several mechanisms of action, IM blocks signal transduction through both the TGF-β receptor and the PDGF-BB receptor. Both TGF-β and PDGF are known to mediate fibrosis. TGF-β mRNA levels are increased in skin, fascia, and striated muscle of patients with NSF (Jimenez et al., Arthritis Rheum 2004, 50:2660-6) but studies have not yet been conducted to examine PDGF expression in tissues from patients with NSF. Gadolinium deposited in skin of patients with NSF might serve as a persistent stimulus for fibrosis, perhaps by inducing production of TGF-β or other profibrotic cytokines. Our observations in these two patients with NSF suggest that IM, perhaps by inhibiting signal transduction through the TGF-β or PDGF-BB receptor, downregulates fibrosis in NSF. Since the skin changes of NSF improved over weeks, during treatment with IM, it would appear that the fibrosis and skin remodeling are dynamic in nature and capable of modulation. Upon discontinuation of IM, skin fibrosis appears to resume, with persistence of gadolinium in tissue in these cases.

This is the first report of clinical and histologic improvement in patients with a fibrosing disorder treated with IM. These initial studies indicate that IM is an effective chronic treatment for NSF and other fibrosing disorders that presently are without effective treatment.

### Conclusions

Imatinib mesylate treatment decreased fibrosis and resulted in the relatively rapid and steady improvement of skin changes and knee joint contractures in patients with stage 5 chronic kidney disease and NSF, despite the persistence of gadolinium in tissue. The recurrence of skin changes after discontinuation of imatinib mesylate suggests that chronic treatment may be required.

### Methods

Skin involvement was assessed using the modified version of the Rodnan skin score (mRSS). The same examiner evaluated each patient at each visit. Seventeen skin sites were evaluated: the face, upper arms, forearms, dorsum of the hands, fingers, chest, abdomen, thighs, forearms, and feet. Each site was graded: 0=normal skin, 1=thickened skin, 2=thickened and unable to pinch, or 3=thickened and unable to move. The maximum total score is 51.

Loss of knee joint extension (flexion contracture) was assessed by measuring maximal passive knee extension, with the patient in a supine position, using a goniometer. The center of the goniometer was placed at the lateral femoral condyle, with the proximal arm positioned along the longitudinal axis of the femur and the distal arm aligned with the long axis of the tibia. Measurements were rounded to the nearest multiple of 5 degrees. Although flexion contractures appeared symmetric in both patients, the knee with the greater flexion contracture at baseline was assessed for change at subsequent visit.

### Example 2

The following studies will be performed to verify the efficacy of imatinib mesylate in reducing cutaneous thickening and tethering in patients with NSF and to assess the safety and tolerability of imatinib mesylate in patients with chronic kidney disease and NSF.

Ten subjects with chronic kidney disease and symptoms, signs and skin biopsy consistent with NSF will undergo thorough history, physical examination, and laboratory testing of blood, as is consistent with clinical standard of care. Baseline modified Rodnan skin score (mRSS), measurement of joint contractures, photography of the skin, skin biopsy, VAS and HAQ will be obtained. Each patient will be followed for one month without intervention. If the patient has spontaneous improvement of mRSS by more than 20%, the patient will not be started on imatinib mesylate; otherwise imatinib mesylate 400 mg daily, oral administration, would be started. The study drug would be continued for the following four months. The drug dose would be reduced to 200 mg daily if patient develops GI intolerance or alopecia. Periodic mRSS, measurements of joint contractures and side-effect profile will be assessed at 1 and 2 months At the end of four months on imatinib mesylate, the drug will be discontinued and a repeat mRSS, measurement of joint contractures, photography of skin, skin biopsy, VAS and HAQ will be obtained. The patient will be followed for another two months after stopping imatinib mesylate and a final mRSS, measurement of joint contractures, VAS and HAQ will be documented.

Because a significance decrease in mRSS was observed in each of 2 patients with NSF, within 2 months of beginning treatment with imatinib mesylate, we expect to observe similar findings among 10 additional patients with chronic kidney disease and NSF.

Data to be collected includes name, medical record number, date of birth, age, gender, history and physical examination including dermatologic exam, photography of skin, complete blood count, electrolytes, BUN/creatinine, calcium, phosphorous, PTH, 25-OH vitamin D, TSAT, Ferritin, ESR, CRP, ANA, anti-scl-70 antibodies, anti-centromere antibodies, PT, PTT, INR, gadolinium exposure (type, dose and timing), modality of dialysis (if applicable), adequacy of dialysis, pre- and post-treatment (at 4 months) and pre- and post-imatinib mesylate treatment skin biopsy.

Subjects over the age of 18 years, which have biopsy-proven NSF will be used. Those with known sensitivity to imatinib mesylate or to any of its components, women who are pregnant or lactating, those with bullous dermatologic disease, with AST/ALT>3×upper limit of normal, or with severe congestive heart failure (NYHA Class Iii or IV) will be excluded from the study.

Biostatistical analysis will be performed on a variety of types of data generated from the study. Specific data variables (e.g. data collection sheets) will be collected. In addition, study endpoints (clinical response to imatinib mesylate at end of treatment (4 months and after 2 months off of treatment) will be analyzed. This includes primary outcome: change in modified Rodnan skin score (mRSS) to assess skin tethering and secondary outcomes. Secondary outcomes include the change in maximal extension of elbows and knees (measured using a goniometer), the change in histologic appearance of skin biopsy, the change in visual analog scale (VAS) for pain, the change in health assessment questionnaire (HAQ) score.

Statistical methods will entail a non-parametric method to evaluate the significance. ANOVA/ANCOVA for continuous variable and Fisher's exact test for categorical variable, student's t-test. Intent to treat analysis with last observation carried forward.

Drug side effects will be studied and minimized wherever possible by adjustment of dosage. Women of childbearing potential will be advised to avoid becoming pregnant. This is because imatinib mesylate was teratogenic in rats when administered during organogenesis at doses ≧100 mg/kg, approximately equal to the maximum clinical dose of 800 mg/day based on body surface area. There are no adequate and well-controlled studies in pregnant women. If a subject becomes pregnant while taking imatinib mesylate, she will be apprised of the potential hazard to the fetus.

Dermatologic toxicities will be noted and minimized. Bullous dermatologic reactions, including erythema multiforme and Stevens-Johnson syndrome, have been reported with use of imatinib mesylate. Concomitant corticosteroids or antihistamine may be beneficial.

Fluid retention and edema will be recorded and minimized. Imatinib mesylate is often associated with edema and occasionally serious fluid retention. This is increased with higher imatinib mesylate dose and age >65 years in the CML studies. Severe superficial edema and fluid retention (pleural effusion, pulmonary edema and ascites) were reported in 1-6% of patients. There have been post-marketing reports, including fatalities, of cardiac tamponade, cerebral edema, increased intracranial pressure, and papilledema in patients treated with imatinib mesylate. Patient's nephrologist should manage the fluid balance.

Gastrointestinal disorders will be monitored and minimized. Imatinib mesylate is sometimes associated with GI irritation and rarely with GI perforation.

Hematologic toxicity will be monitored and minimized. Treatment with imatinib mesylate may be associated with anemia, neutropenia, and thrombocytopenia. Complete blood counts will be performed monthly at the facility where the subject is receiving hemodialysis.

Hepatotoxicity is a possibility. Occasionally severe hepatotoxicity may occur with imatinib mesylate. Liver function (transaminases, bilirubin, and alkaline phosphatase) will be monitored before initiation of treatment and monthly thereafter, for the duration of the study. These will be performed at the facility where the subject is receiving hemodialysis.

Incidences of severe congestive heart failure and left ventricular dysfunction will be recorded. This has been occasionally been reported in patients taking imatinib mesylate. Most of the patients with reported cardiac events have had other co-morbidities and risk factors, including advanced age and previous medical history of cardiac disease. Fluid retention, if it occurs, will be managed by adjusting the dialysis prescription as needed.

Interactions of imatinib mesylate with other drugs will be kept in mind. Drugs that may alter imatinib mesylate plasma concentration such as inhibitors of cytochrome P450 isoenzyme (CYP3A4) may decrease metabolism and increase imatinib mesylate concentrations. Conversely, substances that induce CYP3A4 may increase metabolism and thereby decrease imatinib mesylate concentrations. Drugs that may have their plasma concentration altered by imatinib mesylate: imatinib mesylate increases the concentration of drugs metabolized by CYP3A4, suggesting that imatinib mesylate inhibits CYP3A4. Because warfarin is metabolized by CYP2C9 and CYP3A4, patients who require anticoagulation should receive standard heparin or monitor closely PT/INR on warfarin while on imatinib mesylate.

Patients with NSF are expected to experience improvement in clinical manifestations of NSF, such as skin tethering and thickening and joint contractures. Efficacy of a targeted therapy, such as imatinib mesylate, suggests a potential biological mechanism for the development of fibrosis in NSF. As such, the conclusions of this study will be applicable to other individuals with NSF.

Outcomes monitoring: will consist of evaluating efficacy, adverse events, observation, clinical evaluation and laboratory parameters including hematology, chemistry. The skin biopsy will be performed according to the standard of care.

### REFERENCES

1. Cowper S E, Robin H S, Steinberg S M, Su L D, Gupta S, LeBoit P E. Scleromyxoedema-like cutaneous diseases in renal-dialysis patients. Lancet 2000; 356(9234): 1000-1.
2. Moschella S L, Kay J, Mackool B T, Liu V. Case records of the Massachusetts General Hospital. Weekly clinicopathological exercises. Case 35-2004. A 68-year-old man with end-stage renal disease and thickening of the skin. New England Journal of Medicine 2004; 351(21): 2219-27.
3. Centers for Disease Control and Prevention (CDC). Fibrosing skin condition among patients with renal disease—United States and Europe, 1997-2002. MMWR Morb Mortal Wkly Rep. 2002; 51(2): 25-26.
4. Cowper SE. Nephrogenic systemic fibrosis: the nosological and conceptual evolution of nephrogenic fibrosing dermopathy. Am J Kidney Dis 2005; 46(4): 763-5.
5. Jimenez S A, Artlett C M, Sandorfi N, et al. Dialysis-associated systemic fibrosis (nephrogenic fibrosing dermopathy): study of inflammatory cells and transforming growth factor beta 1 expression in affected skin. Arthritis Rheum 2004; 50(8): 2660-6.
6. Levine J M, Taylor R A, Elman L B, et al. Involvement of skeletal muscle in dialysis-associated systemic fibrosis (nephrogenic fibrosing dermopathy). Muscle Nerve 2004; 30(5): 569-77.
7. Ting W W, Stone M S, Madison K C, Kurtz K. Nephrogenic fibrosing dermopathy with systemic involvement. Arch Dermatol 2003; 139(7): 903-6.
8. Daram S R, Cortese C M, Bastani B. Nephrogenic fibrosing dermopathy/nephrogenic systemic fibrosis: report of a new case with literature review. Am J Kidney Dis 2005; 46(4): 754-9.
9. Cowper S E, Bucala R, LeBoit P E, et al. Case 35-2004: nephrogenic fibrosing dermopathy. New England Journal of Medicine 2005; 352(16): 1723-4.
10. Cowper S E. Nephrogenic fibrosing dermopathy: the first 6 years. Curr Opin Rheumatol 2003; 15(6): 785-90.
11. Swartz R D, Crofford L J, Phan S H, Ike R W, Su L D. Nephrogenic fibrosing dermopathy: a novel cutaneous fibrosing disorder in patients with renal failure. Am J Med 2003; 114(7): 563-72.
12. Grobner T. Gadolinium—a specific trigger for the development of nephrogenic fibrosing dermopathy and nephrogenic systemic fibrosis? Nephrol Dial Transplant 2006; 21(4): 1104-8.
13. Marckmann P, Skov L, Rossen K, et al. Nephrogenic systemic fibrosis: suspected causative role of gadodiamide used for contrast-enhanced magnetic resonance imaging. J Am Soc Nephrol 2006; 17(9): 2359-62.
14. D J Todd, A Kagan, L Chibnik, J. Kay. Cutaneous changes of nephrogenic systemic fibrosis: Predictor of early mortality and association with gadolinium exposure. Arthritis Rheum 2007; 56(10):3433-3441.
15. Cowper S E, Bucala R. Nephrogenic fibrosing dermopathy: suspect identified, motive unclear. Am J Dermatopathol 2003; 25(4): 358.
16. LeBoit P E. What nephrogenic fibrosing dermopathy might be. Arch Dermatol 2003; 139(7): 928-30.
17. Gilliet M, Cozzio A, Burg G, Nestle F O. Successful treatment of three cases of nephrogenic fibrosing dermopathy with extracorporeal photopheresis. Br J Dermatol 2005; 152(3): 531-6.
18. Jiménez S A, Artlett C M, Sandorfi N, Derk C, Latinis K, Sawaya H, Haddad R, Shanahan J C. Dialysis-associated systemic fibrosis (nephrogenic fibrosing dermopathy): Study of inflammatory cells and transforming growth factor β1 expression in affected skin. Arthritis Rheum, 2004; 50: 2660-2666
19. Wataru Ishida W, Swati Bhattacharyya S, Hinchcliff M, Mori Y, Wu M, Takagawa S, Takehara K, Varga J. Novel role of c-Abl tyrosine kinase in profibrotic TGFβ responses: Selective modulation by the anticancer drug imatinib mesylate (Gleevec) [Abstract]. Arthritis Rheum 2006; 54(Supplement).
20. Distler J H W, Astrid Jüngel A, Huber L C, Schulze-Horsel U, Zwerina J, Gay R E, Michel B A, Hauser T, Schett G, Gay S, Distler O. Imatinib mesylate reduces production of extracellular matrix and prevents development of experimental dermal fibrosis. Arthritis Rheum 2007; 56:311-322.

1. A method of treatment and/or prevention of nephrogenic systemic fibrosis (NSF) in a subject in need thereof comprising, administering an effective amount of imatinib mesylate to the subject, to thereby treat or prevent the nephrogenic systemic fibrosis.

2. The method of claim 1 further comprising selecting a subject that has been diagnosed with NSF, has one or more symptoms associated with NSF, or is at increased risk for NSF, prior to administering the imatinib mesylate.

3. The method of claim 2, wherein the symptom associated with NSF is rapidly progressive skin hardening, skin tethering, hyperpigmentation, joint flexion contracture, extracutaneous fibrosis involving the heart, lungs, diaphragm, skeletal muscle, liver, genitourinary tract, or central nervous system, and combinations thereof.

4. The method of claim 1 or 2, wherein the subject has chronic kidney disease, is undergoing hemodialysis, is in renal failure, has been exposed to gadolinium, or any combination thereof.

5. The method of claim 4, wherein the gadolinium is in the form of a gadolinium-containing contrast.

6. The method of claim 1 or 2, wherein the subject also undergoes another therapy to treat or prevent nephrogenic systemic fibrosis, chronic kidney disease, or renal failure.

7. The method of claim 6, wherein the other therapy is extracorporeal photopheresis.

8. The method of claim 6, wherein the other treatment is administration of pentoxyphyline.

9. The method of claim 1 or 2, wherein treatment results in softening of previously hardened skin, and/or increased mobility of skin, and/or improved joint mobility.

10. The method of claim 1 or 2 further comprising administration of a corticosteroid and/or an antihistamine.

11. The method of claims 1-10, wherein administration is systemic.

12. The method of claim 11, wherein administration is oral.

13. The method of claim 11 or 12, wherein between about 200 mg and 600 mg of imatinib mesylate is administered daily.

14. The method of claim 13, wherein about 600 mg, 400 mg, or 200 mg of imatinib mesylate is administered daily.

15. A pharmaceutical composition comprising imatinib mesylate and a pharmaceutically acceptable carrier.

16. The pharmaceutical composition of claim 15, that is formulated for oral administration.

17. The pharmaceutical composition of claim 15 or 16, that contains about 100-800 mg imatinib mesylate.

18. The pharmaceutical composition of claim 17, that contains about 200-600 mg imatinib mesylate.

19. The pharmaceutical composition of claim 17, that contains about 200-400 mg imatinib mesylate.

20. The pharmaceutical composition of claim 17, that contains about 200 mg, 400 mg, 600 mg, or 800 mg imatinib mesylate.
